(19) United States
(12) Patent Application Publication
(10) Pub. No.: US 2021/0375446 A1
(43) Pub. Date: Dec. 2, 2021

(54) SYSTEMS FOR MONITORING ROOMS IN A MEDICAL FACILITY

(71) Applicant: General Electric Company, Schenectady, NY (US)

(72) Inventors: John Page, Verona (US); Brandon Henak, Madison (US); Kevin Tissot, Brooklyn (US); Guy Vesto, Kildeer (US)

(73) Assignee: General Electric Company, Schenectady, NY (US)

(21) Appl. No.: 17/405,750

(22) Filed: Aug. 18, 2021

Related U.S. Application Data
(63) Continuation-in-part of application No. 15/994,298, filed on May 31, 2018.

(51) Int. Cl. G16H 40/60; G16H 40/20; G16H 40/40

(52) U.S. Cl. CPC G16H 40/60; G16H 40/40; G16H 40/20

(57) ABSTRACT

A system for monitoring room use in a medical facility. The system includes medical devices having sensors that produce machine data corresponding to operation of that medical device. A central processing module analyzes the machine data to identify portions corresponding to clinical cases via at least one of: detecting changes in operational status within the medical devices, detecting changes to devices settings within the medical devices, and detecting a delivery or consumption of anesthetic agent by the medical devices. The central processing module further analyzes the identified portions of the machine data corresponding to the clinical cases to identify procedural events, then automatically determines a current use status for the monitored rooms based on the procedural events identified. A display device communicates with the central processing module and displays the current use status determined for each of the monitored rooms.

### CROSS REFERENCE TO RELATED APPLICATIONS

This application is a continuation-in-part of U.S. patent application Ser. No. 15/994,298, filed May 31, 2018, which is incorporated herein by reference in its entirety.

### FIELD

The present disclosure generally relates to systems for monitoring rooms in a medical facility.

### BACKGROUND

The present disclosure is related to the field of data processing and analytics. More specifically, the present disclosure is related to predictive scheduling based upon collected data and processing of medical device data.

Medical devices generate large amounts of data from sensors and inputs that are used in real-time on the device to display relevant information or to provide alerts to users of the device. In some settings, this data is made available to external systems through proprietary or standards-based data messaging. Examples of these systems include hospital information systems (HIS) and/or electronic health records (EHR). HIS and EHR systems collect and process a variety of information, including patient data. HIS systems seek to coordinate the administrative, financial, legal needs of a hospital system, which may include the management of patient data or particular departments within a hospital system or network, for example laboratory services and patient imaging. EHR systems seek to collect and coordinate health information generated both on a patient by patient basis as well as population information. In either case of HIS or EHR systems, these currently available systems only connect to medical devices in a limited manner and to the extent that information directly from medical devices is incorporated into these systems, such information is typically the physiological data directly acquired from such medical devices, e.g. heart rate, pulse rate, blood oxygenation, blood pressure, temperature, respiration rate, etc.

However, medical devices as typically used in hospitals produce significantly more data than merely the monitor physiological parameters of a patient. Therefore, new systems and methods are desirable to collect and process this medical device data and to incorporate information gained from these systems into hospital system workflows and decision making.

### BRIEF DISCLOSURE

This Summary is provided to introduce a selection of concepts that are further described below in the Detailed Description. This Summary is not intended to identify key or essential features of the claimed subject matter, nor is it intended to be used as an aid in limiting the scope of the claimed subject matter.

One embodiment of the present disclosure generally relates to a system for monitoring a use status of monitored rooms in a medical facility. The system includes a plurality of medical devices located in the monitored rooms, where each of the plurality of medical devices comprises at least one sensor that produces machine data corresponding to operation of that medical device, and where the plurality of medical devices are configured to output a plurality of streaming time series of medical device data that includes the machine data produced by the at least one sensor. A central input-output module communicates with the plurality of medical devices and receives in real time or near real time the machine data therefrom. A central processing module communicates with the central input-output module, where the central processing module is configured to analyze the machine data to identify portions corresponding to occurrences of clinical cases via at least one of: detecting changes in operational status within the plurality of medical devices, detecting changes to devices settings within the plurality of medical devices, and detecting a delivery or consumption of anesthetic agent by the plurality of medical devices, where the central processing module is further configured to analyze the identified portions of each of the machine data corresponding to the clinical cases to identify procedural events within the identified portions, and where the central processing module is further configured to automatically determine a current use status for each of the plurality of monitored rooms of the medical facility based on the procedural events identified within the machine data. A display device communicates with the central processing module and displays the current use status for each of the plurality of monitored rooms determined thereby. In certain examples, the central processing module is further configured to compare the current use status determined for each of the plurality of monitored rooms to expected use status corresponding thereto, and to generate an alert when the current use status is inconsistent with the expected use status.

In certain examples, the plurality of streaming time series of medical device data includes a plurality of time series of medical device data streams from a single device of the plurality of medical devices, and the central processing module is configured to concurrently analyze the identified portions of each of the plurality of time series of medical device data streams corresponding to the clinical case from the single device to identify the procedural events and the current use status of the monitored room comprising the single medical device.

In certain examples, the display device displays the use status for each of the plurality of monitored rooms in real time. In further examples, the current use status further includes a current procedure phase identified by the central processing module. Further examples include a memory module in communication with the central processing module, and further include clinical case models stored within the memory module that include estimated times for completing the clinical cases, where the central processing module is further configured to estimate a time at which the current use status will end based on which of the estimated times corresponds to the clinical case identified.

In certain examples, the machine data is one or more of a compressor speed, a compressor cycle time, an internal pressure, a voltage, a clock speed, and a temperature. In further examples, the machine data is received at or near a frequency at which the machine data is produced by the medical device. Further examples include a memory module in communication with the central processing module, and further include clinical case models stored within the memory module that include estimated times for completing the clinical cases, where the central processing module is further configured to determine a current procedure phase of the current use status from the procedural events, where the display device is configured to provide a real-time visual output of the current procedure phase, and where the central processing module is further configured to determine a predication of when a next procedure phase will begin. In further examples, the central processing module is further configured to provide the prediction to a scheduling system for the medical facility. In further examples, the central processing system is further configured to, based upon the prediction, communicate to the scheduling system for the medical facility to reserve a subsequently required resource. In further examples, the procedure phases include induction, maintenance, and emergence. In further examples, the medical device data further comprises anesthetic agent flow rate, oxygen flow rate, expiration pressure, and expired gas concentration. In further examples, the plurality of time series medical device data comprises an anesthetic agent flow rate, and where the central processing module is configured to analyze the plurality of time series medical device data by identifying a start of a procedure and an induction phase based upon an increase in the anesthetic agent flow rate from a zero flow rate in the time series medical device data, and by identifying an emergence phase based upon an anesthetic agent flow rate of zero combined with an increased oxygen flow rate in the time series medical device data.

Certain examples further include a memory module in communication with the central processing module, and further include a plurality of procedure models stored within the memory module, where the central processing module, in analyzing the plurality of time series of medical device data, is configured to compare the plurality of time series of medical device data to the plurality of procedure models to identify the current use status of each of the monitored rooms.

Certain examples further include a memory module in communication with the central processing module, and further include configuring the central processing module to generate and store in the memory module procedure models as a composite of a plurality of previously recorded and annotated time series of medical device data from the plurality of time series of medical device data. Further examples include determining an estimate of when a current use status will end by comparing the streaming time series medical device data to the procedure models.

Certain examples further include a memory module in communication with the central processing module, and a plurality of case identification rules for applying to the plurality of streaming time series of medical device data, the plurality of case identification rules being stored in the memory module, and the central processing module being configured to apply the plurality of case identification rules and to produce an indication of a type of procedure in which the medical device is used.

In certain examples, the central processing module is further configured to aggregate the determined current use status for each of the plurality of monitored rooms of the medical facility over a predetermined time period, and to output a summary of the determined use statuses for the predetermined time period. In further examples, the central processing module is further configured to calculate a utilization rate for each of the monitored rooms based upon the determined use statuses for the predetermined time period.

In certain examples, the central processing module is further configured to determine at least one daily use metric from the plurality of streaming time series of medical device data from at least one medical device located in a given monitored room among the monitored rooms, wherein the at least one daily use metric comprises a clinical start time, a clinical end time, a utilization rate, a case volume, or an average turnover time.

### BRIEF DESCRIPTION OF THE DRAWINGS

FIG. 1 is a schematic view of an exemplary system for monitoring a use status of a monitored room according to the present disclosure.

FIG. 2 an information process flow depicting inputs and outputs of the system shown in FIG. 1.

FIG. 3 is a system diagram of an exemplary embodiment of a system for medical device data collection and processing.

FIG. 4 depicts an exemplary embodiment of a method of medical device data processing.

FIG. 5 depicts an exemplary embodiment of a method of medical device data curation and learning.

FIG. 6 depicts an exemplary embodiment of a monitored room status dashboard.

FIG. 7 depicts an exemplary embodiment of a monitored room status dashboard.

FIG. 8 is a system diagram of an exemplary embodiment of a system for medical device data collection and processing.

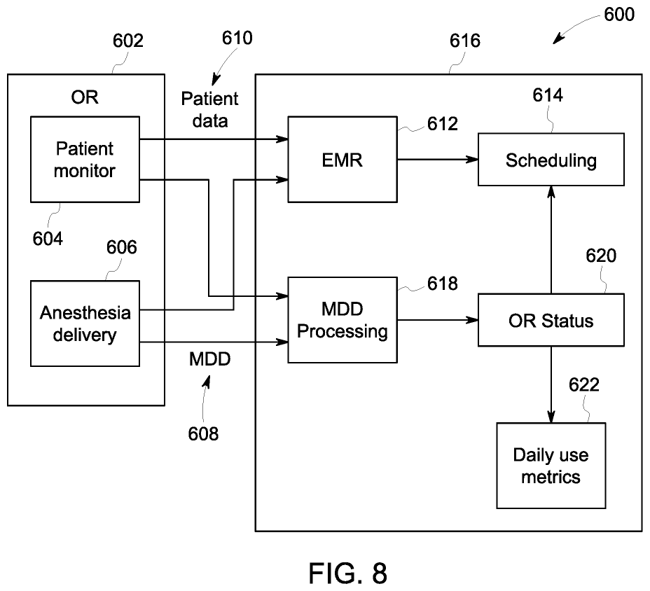

FIG. 9 depicts an exemplary embodiment of an operating room summary dashboard.

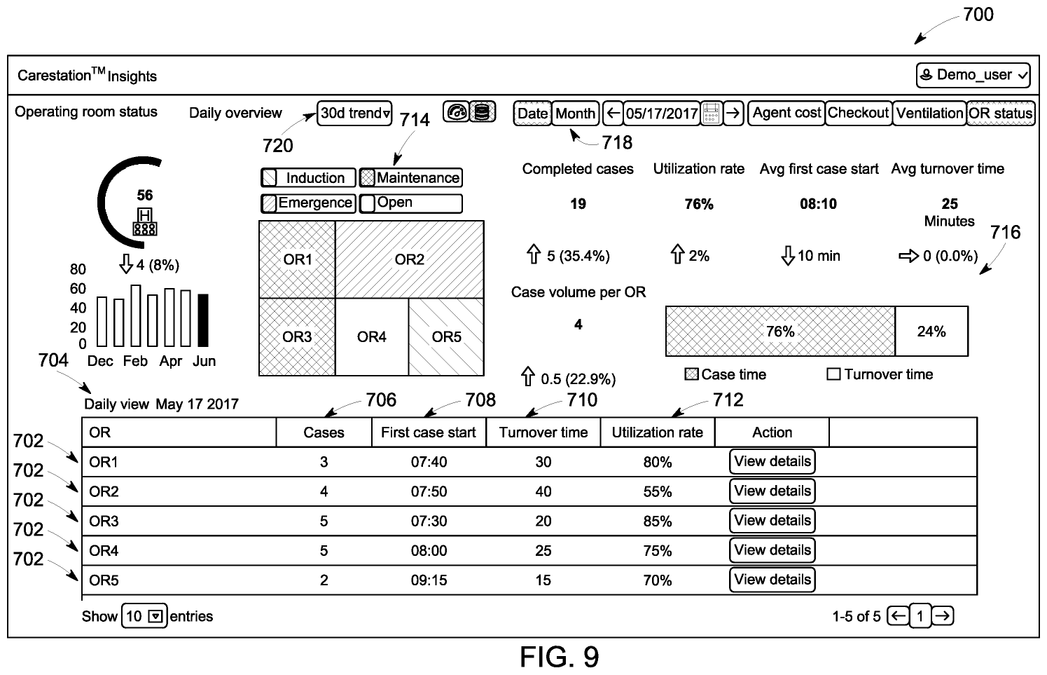

FIG. 10 is a flow chart that depicts an exemplary embodiment of a method of monitoring a use status of a medical facility.

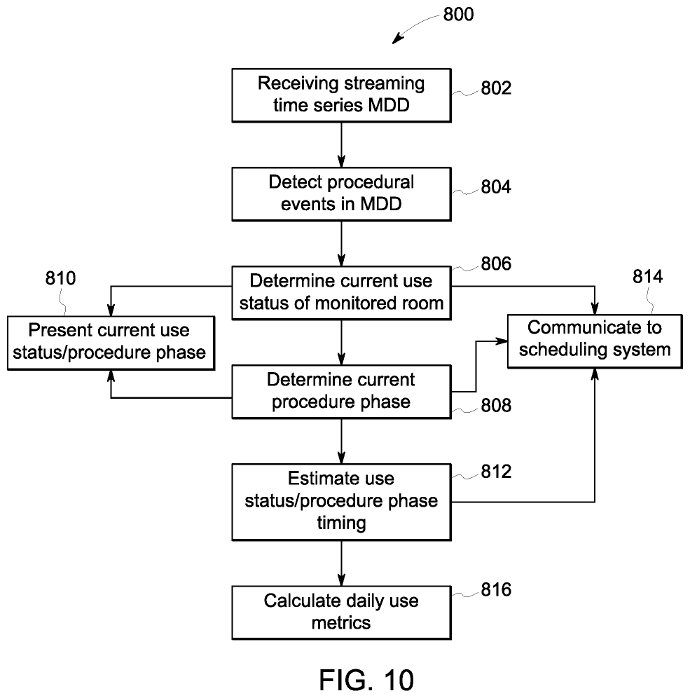

### DETAILED DISCLOSURE

Embodiments of systems and methods as disclosed herein operate to collect and process a wide variety of medical device data. Medical device data includes physiological data that is acquired from a patient by a medical device and machine data collected internally from the medical device itself. Machine data can include alarms, device status, settings, messages, and measured operational data. Machine data may further include settings and values that represent specific actions taken with the medical device for example, in response to automated controls or due to clinician inputs. For example, in an anesthesia delivery machine, this may include changes to oxygen and/or anesthetic agent concentrations. The machine data may further include clinical and/or technical alarms initiated by the medical device or device diagnostic information. Still further examples of the machine data include proactive or predictive service alerts from the medical device, maintenance checkout information and/or processor clock cycle signals or power signals or other operational signals from various components of the medical device indicative that the medical device is turned on, in use, in operation, held in a stand by condition, or turned off.

This machine data can be collected in time series format as provided from the medical devices themselves. As used herein, the time series format of the medical device data can include waveforms, binary data, numeric data, and/or textual data in a time series format. Embodiments of the systems and methods as disclosed herein receive the medical device data from the medical devices at a frequency similar to the frequency at which it is produced by the medical device. In embodiments, this increased velocity of the received data and the monitoring and analysis of medical device machine data can enable improved monitoring systems and method as disclosed herein. As described in further detail herein embodiments of systems and methods support high speed data ingestion, enrichment, normalization, and data curation of the medical device data. The medical device data can undergo real time analysis and further enrichment of the data with event detection and notation. While all of the medical device data can be saved for retrospective and automated machine learning and analysis, event detection and notation can be used to create further exemplary files of medical device data stemming from particular events or conditions which can be used as exemplary or case study data for further analysis.

In embodiments, machine learning and data analysis of the collected medical device data can be used to develop and refine control algorithms, early detection/predictive algorithms, and/or alarm algorithms to improve clinician use of the medical devices, hospital workflow, operation or alarm settings of the medical devices themselves, or event detection within the system.

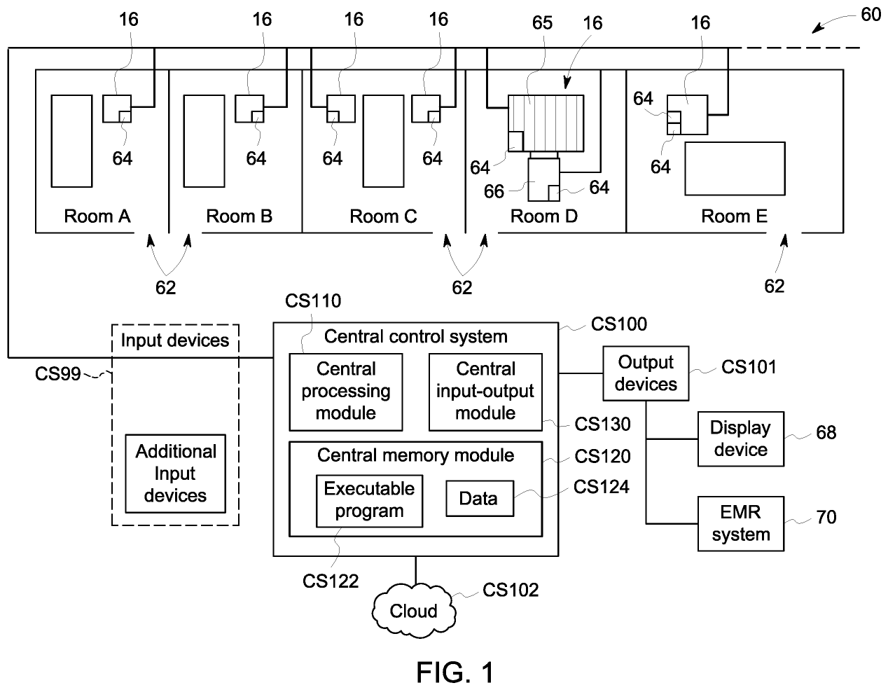

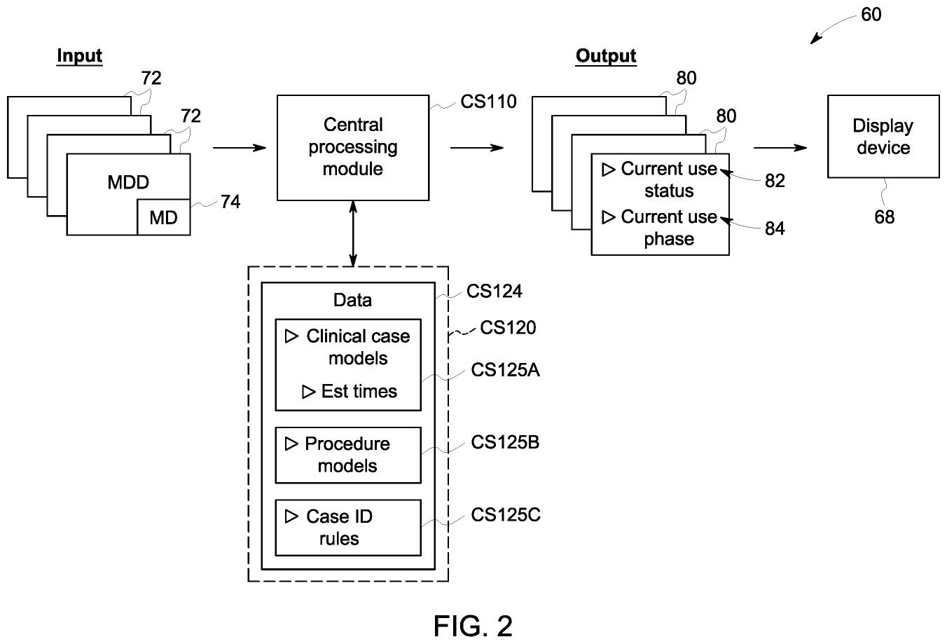

FIG. 1 depicts an exemplary system 60 for monitoring a use status of a monitored room 62 within a medical facility. In the example shown, there are five monitored rooms 62 labeled Room A through Room E, which here correspond to three patient rooms, an imaging suite (e.g., containing a CT scanner), and a surgical suite, respectively. Each of the monitored rooms 62 includes at least one medical device 16 each having at least one sensor 64 therein. Exemplary medical devices 16 include patient monitoring devices, ventilators, anesthesia machines, and CT scanners (shown here as the scanner 65 and corresponding table 66 as shown in the art), for example. As shown in FIGS. 1 and 2, the medical devices 16 are configured to output streaming time series of medical device data 72 that includes machine data 74 produced by the sensors 64, whereby the machine data 74 corresponds to operation of that medical device 16. Additional information regarding medical devices, medical device data, machine data, and sensors is provided below.

Returning to FIG. 1, the system 60 further includes a central control system CS100 having a central processing module CS110, central memory module CS120, and central input/output (I/O) module CS130, which may be implemented as hardware, software, or combinations thereof as discussed further below. The central input/output (I/O) module CS130 communicates with the medical devices 16 and receives in real time or near real time the machine data 74 therefrom. The central input/output (I/O) module CS130 may also communicate with additional input devices CS99.

The central processing module CS110 communicates with the central input/output (I/O) module CS130 and is configured to analyze the machine data 74 to identify portions corresponding to occurrences of clinical cases via at least one of: detecting changes in operational status within the plurality of medical devices, detecting changes to devices settings within the plurality of medical devices, and detecting a delivery or consumption of anesthetic agent by the plurality of medical devices. The central processing module CS110 is further configured to analyze the identified portions of each of the machine data 74 corresponding to the clinical cases to identify procedural events within the identified portions. The central processing module CS110 then automatically determines a current use status for each of the monitored rooms 62 based on the procedural events identified within the machine data 74. A display device 68 (such as a computer monitor, tablet, or smart phone) communicates with the central processing module CS110 and displays the current use status for each of the monitored rooms 62 as determined. The central control system CS100 may also or alternatively communicate with other output devices CS101, such as an EMR system 70 presently known in the art. Additional information regarding the analysis by the central processing module CS110 is provided below.

In certain examples, the central processing module CS110 is further configured to compare the current use status determined for each of the plurality of monitored rooms to expected use status corresponding thereto, and to generate an alert when the current use status is inconsistent with the expected use status. The expected use status for each of the plurality of monitored rooms may be stored as a database within the central memory module CS120, based on empirically derived times for each procedure and/or the like, for example.

Additional information regarding the central control system CS100 is provided herein and shown in FIG. 1. Certain aspects of the present disclosure are described or depicted as functional and/or logical block components or processing steps, which may be performed by any number of hardware, software, and/or firmware components configured to perform the specified functions. For example, certain embodiments employ integrated circuit components, such as memory elements, digital signal processing elements, logic elements, look-up tables, or the like, configured to carry out a variety of functions under the control of one or more processors or other control devices. The connections between functional and logical block components are merely exemplary, which may be direct or indirect, and may follow alternate pathways.

In certain examples, the central control system CS100 communicates with each of the one or more components of the system 60 via a communication link CL, which can be any wired or wireless link. The central control module CS100 is capable of receiving information and/or controlling one or more operational characteristics of the system 60 and its various sub-systems by sending and receiving control signals via the communication links CL. In one example, the communication link CL is a controller area network (CAN) bus; however, other types of links could be used. It will be recognized that the extent of connections and the communication links CL may in fact be one or more shared connections, or links, among some or all of the components in the system 60. Moreover, the communication link CL lines are meant only to demonstrate that the various control elements are capable of communicating with one another, and do not represent actual wiring connections between the various elements, nor do they represent the only paths of communication between the elements. Additionally, the system 60 may incorporate various types of communication devices and systems, and thus the illustrated communication links CL may in fact represent various different types of wireless and/or wired data communication systems.

The central control system CS100 may be a computing system that includes a processing system CS110, central memory module CS120, and central input/output (I/O) module CS130 for communicating with other devices, such as input devices CS99 and output devices CS101, which may communicate with and/or be provided as a cloud 102. The processing system CS110 loads and executes an executable program CS122 from the central memory module CS120, accesses data CS124 stored within the central memory module CS120, and directs the system 60 to operate as described in further detail below.

The processing system CS110 may be implemented as a single microprocessor or other circuitry, or be distributed across multiple processing devices or sub-systems that cooperate to execute the executable program CS122 from the central memory module CS120. Non-limiting examples of the processing system include general purpose central processing units, application specific processors, and logic devices.

The central memory module CS120 may comprise any storage media readable by the processing system CS110 and capable of storing the executable program CS122 and/or data CS124. As shown in the example of FIG. 2, the data CS124 may include clinical case models CS125A, in some examples including estimated times for completing the clinical cases (which may be provided from empirical study and/or target goals, for example). The data CS124 may further include procedure models CS125B and/or case identification rules CS125C for reference and comparison by the central processing module CS110, as discussed further below. Once processing is completed, the central processing module CS110 may output a summary 80 of the use of each of the monitored rooms 62, which in certain examples includes a current use status 82 and also a current use phase 84. This output may then provided to a display device 68, for example, such as a centralized monitor or a cloud-based scheduling tool.

The central memory module CS120 may be implemented as a single storage device, or be distributed across multiple storage devices or sub-systems that cooperate to store computer readable instructions, data structures, program modules, or other data. The central memory module CS120 may include volatile and/or non-volatile systems, and may include removable and/or non-removable media implemented in any method or technology for storage of information. The storage media may include non-transitory and/or transitory storage media, including random access memory, read only memory, magnetic discs, optical discs, flash memory, virtual memory, and non-virtual memory, magnetic storage devices, or any other medium which can be used to store information and be accessed by an instruction execution system, for example.

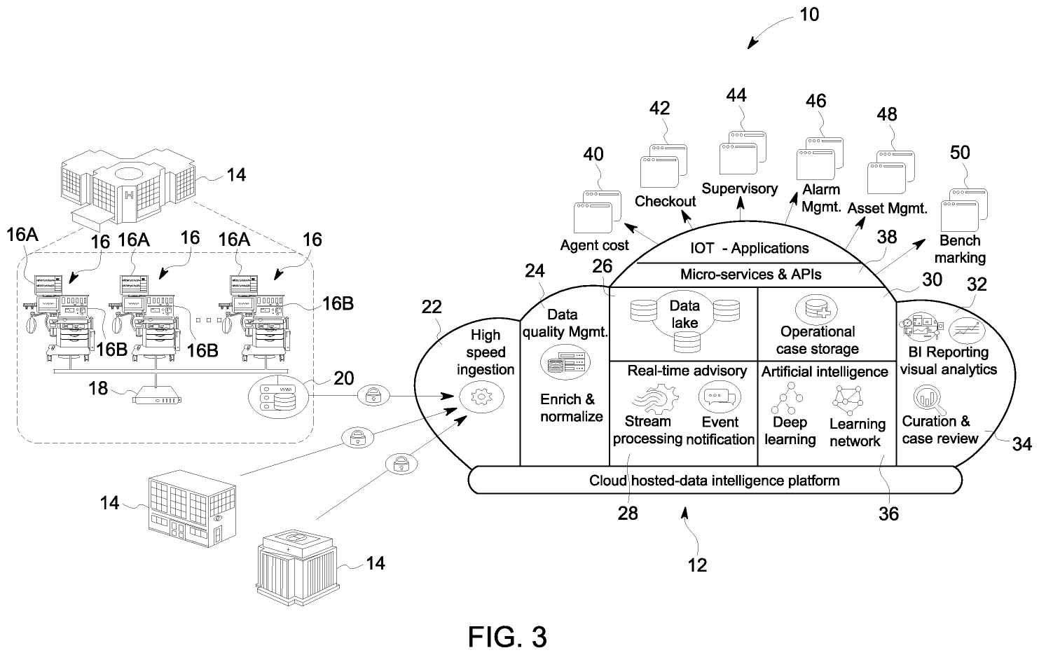

FIG. 3 depicts an exemplary embodiment of a system 10 for medical device data collection and processing. The system 10 includes a medical device data (MDD) processing system 12. The MDD processing system 12 can be implemented in a variety of hardware and/or software implementations, it should be noted that such implementations are not considered to be limiting. For example, it is contemplated that any or all of the MDD processing system 12 may be embodied exclusively in hardware, exclusively in software, exclusively in firmware or in any combination of hardware, software, and/or firmware. While the following describes exemplary methods and systems, the examples provided herein are not the only way to implement such methods and systems.

In embodiments wherein any of the claims are read to cover an entirely software and/or firmware implementation, in any embodiment, at least one of the elements is hereby expressly defined to include a tangible and non-transient computer readable medium. As used herein, the term tangible computer readable medium is expressly defined to include any type of computer readable storage and to exclude propagating signals. Additionally or alternatively, the example methods and systems may be implemented using coded instruction (e.g., computer readable instructions) stored on a non-transitory computer readable medium such as a flash memory, a read-only memory (ROM) a random-access memory (RAM), a cache, or any other storage media in which information is stored for any duration (e.g. for extended period time periods, permanently, brief instances, for temporarily buffering, and/or for caching of the information). As used herein, the term non-transitory computer readable medium is expressly defined to include any type of computer readable medium and to exclude propagating signals.

In exemplary and non-limiting embodiments of the medical device data processing system 12, the system 12 is implemented by one or more networked processors or computing devices. Processing system 12 may be implemented in a cloud computing platform and/or infrastructure. Memory and processors as referred to herein can be standalone or integrally constructed as part of various programmable devices, including for example, computers or servers. Computer memory of computer readable storage mediums as referenced herein may include volatile and non-volatile or removable and non-removable media for a storage of electronic-formatted information such as computer readable program instructions or modules of computer readable program instructions, data, etc. that may be stand-alone or as part of a computing device. Examples of computer memory may include, but are not limited to RAM, ROM, EEPROM, flash memory, CD-ROM, DVD-ROM or other optical storage, magnetic cassettes, magnetic tape, magnetic disc, or other magnetic storage devices, or any other medium which can be used to store the desired electronic format of information and which can be accessed by the processor or processors or at least a portion of a computing device.

The MDD processing system 12 is communicatively connected to at least one hospital network 14. Such communicative connections as well as the hospital network itself may include, but are not limited to, a wide area network (WAN); a local area network (LAN); the internet; wired or wireless (e.g. optical, Bluetooth, radio frequency (RF) network; a cloud-based computer infrastructure of computers, routers, servers, gateways, etc.; or any combination thereof associated therewith that allows the system or portions thereof to communicate with one or more computing devices.

The hospital network 14 may exemplarily be a network associated with a portion of a hospital, for example a surgery unit or department of a hospital, or may be more broadly located across medical devices of an entire hospital. It further will be recognized that while some embodiments and implementations of the systems and methods as disclosed herein may seek to operate on a single hospital or unit of a hospital, still other embodiments may connect a plurality of hospital networks, including hospitals currently owned or operated or otherwise affiliated with one another. In still further embodiments while individual hospitals or groups of hospitals may use the MDD processing system 12, the MDD processing system 12 receives and processes information from a plurality of hospital networks including those unaffiliated with one another at the same time.

As depicted in FIG. 3, the hospital network 14 includes a plurality of medical devices 16. The medical devices 16 exemplarily include physiological monitoring devices 16a as well as patient therapy devices 16b. Physiological monitoring devices 16a may include, but are not limited to heart rate monitors, blood pressure oxygenation monitors, respiration monitors, ECG monitors, EEG monitors, or EMG monitors. An exemplary embodiment of an anesthesia delivery machine will be used for discussion purposes as the medical device, and more specifically as the patient therapy device 16b, although it will be recognized by a person of ordinary skill in the art that other devices, including, but not limited to patient respiratory assistance devices or dialysis machines may be further non-limiting examples of patient therapy devices. However, it will be recognized that therapy devices may also include capabilities to not only deliver patient therapy, but also to measure physiological parameters of a patient. For example, embodiments of anesthesia delivery machines may include gas analysis modules operable to measure gas concentrations expired by the patient. In other exemplary embodiments, imaging devices, including, but not limited to X-ray, CT, MRI, and ultrasound devices may be examples of medical devices 16 as contemplated within the present disclosure. Still further examples of medical devices may include video and/or audio recording devices.

In an exemplary embodiment, a limited version of the MDD processing system 12 as described herein may be implemented locally, exemplarily as an anesthesia delivery management system 18. In such an embodiment, the anesthesia delivery management system 18 may operate to collect medical device data from a plurality of anesthesia delivery machines 16b for example, among other things as described herein, to monitor anesthesia agent use between anesthesia delivery machines and across procedures performed by those machines in an effort to visualize anesthetic agent consumption and use as well as to quantify, monitor, and evaluate trends across all of the anesthesia delivery machines in the hospital or surgical unit. In still further embodiments as disclosed herein, the medical device data can be used to manage the schedule and/or maintenance of medical devices used in the hospital or surgical unit.

The medical devices 16 are communicatively connected to one or more gateway devices 20. Gateway devices 20 may exemplarily be edge processing devices, cloud processing devices or internet gateways. The gateway 20 may exemplarily be an internet of things (IOT) gateway which facilitates a secure communications link between the medical devices 16 at the hospital network 14 with the servers, processors, and computer readable media implementing the MDD processing system 12. In exemplary embodiments, the gateway 20 may communicate directly with one or more of the medical devices 16, or may communicate with the medical devices 16 through an intermediate network, for example, the anesthesia delivery management system 18 or another medical device data system or network.

The gateway 20 receives the medical device data as time series data for any of the medical device data available from the medical devices. Exemplary embodiments as described herein will focus on machine data, except where specifically noted, although it will be recognized that other forms of medical device data may be used in similar manners as disclosed with respect to these exemplary embodiments. As noted above, the data streams of machine data are available in time series format as acquired from the medical devices and may include, but are not limited to time series information of alarms, device status, device settings, messages, and measured data. In embodiments, the medical devices may be equipped with sensors that improve the self-awareness of the medical device, e.g. sensors that monitor the function, inputs and/or outputs of various components of the medical device itself. Many such sensors are already incorporated into medical devices such as to measure compressor speeds and/or cycle times, internal pressures, voltages, clock speeds, or temperatures, or other sensors as will be recognized by a person of ordinary skill in the art or as disclosed in further detail herein.

The gateway 20 encrypts the time series formatted data and the encrypted data is transmitted using known wired and/or wireless communication techniques for encrypted data to the server, processors, and data storage carrying out the medical device data processing system 12. The gateway 20 continuously transmits de-identified medical device machine data in time series format over an encrypted communication channel to a high speed data ingestion module 22 of the MDD processing system 12. While the exemplary embodiment described herein may reference de-identified data, it will be recognized that other embodiments may use patient-identified data with appropriate considerations taken for handling patient data. The high speed data ingestion module 22 takes in the real time streams of medical device data. The data ingestion can be performed automatedly and preprocess the received streams of real time data in the time series for later processing by the MDD processing system 12. The high speed ingestion module 22 can receive concurrent data streams from multiple connected devices across multiple sites at a high incoming velocity, for example at or near the frequency at which medical devices can output machine data. In exemplary embodiments the high speed ingestion module 22 is scalable to continue to ingest increased bandwidth of medical device data without significant decrease in ingestion speeds.

The high speed ingestion module 22 takes the time series medical device data from the medical devices of one or more hospital networks and formats it for further processing by a data quality management module 24. In exemplary and non-limiting embodiments, the high speed injection module 22 supports open standard such as ASTMF 276I or integrated clinical environmental (ICE). The data quality management module 24 exemplarily normalizes, enriches, and tags the data streams without negatively impacting data latency. In a healthcare environment, a variety of healthcare information products and/or systems may be used to provide medical services, collect medical data, conduct medical exams, etc. Such products and/or systems include the Centricity® suite of products and/or systems as offered by GE Healthcare®. However, many healthcare information systems operate using various messaging standards (e.g., Health Level 7 International (HL7 V2.x/v3), Clinical Document Architecture/Continuity Of Care Document (CDA/CCD), American Society for Testing Materials (ASTM), Digital Imaging and Communications in Medicine (DICOM), etc.)) and various standards and/or protocols (e.g., cross-enterprise document sharing (XDS.A/B) cross-enterprise document media interchange (XDM) cross-enterprise document reliable interchange (XDR), patient identifier cross-referencing/patient demographics query (PIX/PDQ) patient administration management (PAM), query for existing data (QED), national counsel for prescription drug programs (NCPDP), etc.)) that make system integration and/or communication more difficult. Thus, normalization may include reformatting of medical data to a consistent or compatible format for use within the MDD processing system 12. In an exemplary embodiment, the medical device data may be normalized into the ISO/IEEE 11073-10101 nomenclature and its extensions. In a still further exemplary embodiment, the data quality management module 24 can normalize the streams of incoming time series data by converting units of measure. The data quality management module 24 can further operate to identify and tag various types of medical device data, locations from which the medical device data was received, or time series data streams originating from the same medical device. These tags can be used as further detailed herein to identify and analyze groups of streams of time series data.

In an exemplary embodiment, the data quality management module 24 normalizes the received incoming data by transforming and/or translating the clinical data streamed from the source healthcare system or device into a canonical data model with associated metadata. The processed medical device data is stored in a data lake 26 which is exemplarily implemented in computer readable storage embodying capability to store terabytes of data. The data lake 26 is a long-term computer storage repository that holds large amounts of raw data in the it's native format until the data is needed. The native format may include the time series data from the medical devices which may be in waveform or binary format, audio data, image data, and/or video data. In embodiments, this can help to facilitate the ingestion of the data that while may not be processed in real time, may still be taken in in real time or near real time and instead stored in the data lake until further needed. This may be facilitated by identifying particular data streams and limiting the processing of those data streams, for example by the data quality management module 24, if it is known at that time that such data stream is not being used in real time analysis. In an exemplary embodiment, the data quality management module 24 may not convert the data to a canonical data model but may still attempt to tag, enrich, or index the data to facilitate later retrieval of that data in a standardized way from the data lake 26.

In a still further embodiment, portions of the data that are stored in the data lake 26 may also be additionally stored in a graph database which may be a separate database residing on the same computer readable storage, or may be embodied on separate computer readable storage from the data lake 26. The graph database may receive the data streams of which it is known that the system may analyze trends in that data stream. The graph database may store the streams of data in a time series format in a way that facilitates trending of the data over time and appending the data with events either identified in the data itself, in one or more of the other data streams, or received by the system from an external source. These events may include, but are not limited to medical device or clinician actions, clinical events, situations, or complications that arise during the medical procedure. The graph database may later be used by a clinician or technician to identify further relationships between trends and the data streams with other analysis as disclosed herein.

At the same time that the data is stored in the data lake 26, the enriched and normalized medical device data is provided to a stream processing engine 28 that uses artificial intelligence capability as described in further detail herein. The stream processing engine 28 identifies cases and events in the time series streams of medical device data. Identified clinical cases can be stored in an operational case database 30. Clinical cases may exemplarily include surgical and intensive care unit (ICU) cases. The clinical cases can be exemplarily identified by the medical device used and the timing of the medical data in the time series of the medical device data. For example, a time series of medical device data from an anesthesia delivery machine showing a change in status turning the machine and followed by changes to device settings and delivery and/or consumption of anesthetic agent all indicate that a clinical case has begun or is ongoing.

As noted above, the streams of time series medical device data originating from the same medical device or from the same location in a hospital may be tagged or otherwise identified as being related. These tags can be used to simultaneously analyze related data streams or combine analysis of related data streams to identify clinical cases. For example, a device status data stream analysis may be combined with a user input data stream, device setting data stream, and operational data streams to identify when the device is used and how it is used in the clinical case. This information may help to distinguish between a maintenance or checkout of the medical device by a technician from the use of the device for clinical case.

The analysis of the data streams of multiple medical devices, particularly those identified as being related or co-located can further be used to identify clinical cases. For example, coordinated or similar actions in data streams of an anesthesia delivery device and a related patient monitoring device, and/or respiratory support device and/or imaging device, etc. can further be used to identify that these devices are being used together for a clinical case. In still further embodiments, the streaming time series medical device data may be combined with information regarding scheduled clinical cases to help to further identify when and how the medical devices are used during clinical cases.

In embodiments, knowledge of a scheduled use of the medical device (e.g. anesthesia delivery machine) can be used to further identify clinical cases in the streams of medical device data. For example, input or received knowledge regarding a type and time of a scheduled procedure may help to identify the start and end of the clinical case in particular streams of medical device machine data. In an embodiment, a known schedule of use for the medical device can help to identify clinical cases from maintenance or calibration actions which may similarly require powering up and at least partial operation of the medical device.

The medical device data associated with the actions by the anesthesia delivery device and/or other medical devices during the identified clinical case are stored in the operational case database 30. In an example, the identification of the clinical case is stored along with the other time series streams of medical device data from that anesthesia delivery machine as well as time series streams of medical device data from any physiological monitors and/or other medical devices associated with the use of that anesthesia delivery machine. In another exemplary embodiment as described in further detail, a clinical case summary with links or identifiers to the associated time series medical device data stored in the data lake 26 can be created and stored in the operational case database 30.

In an embodiment, prior to storing the clinical cases in the clinical case database 30, the clinical cases may be classified or profiled which is a technique used for data curation. The profiling of the clinical cases may be based upon in part, the information in the clinical case summary, and as described in further detail herein, may be used to group the clinical cases into groups, for example normal cases, edge cases, and outlier cases. These determinations may be made in view of a comparison between the time series data in the clinical case against normal distributions of the same type of time series machine data in other similar clinical cases. Edge cases may be identified as borderline or ambiguous cases, not clearly defined as either normal or an outlier. In a merely exemplary embodiment, for a particular measured value or occurrence, a distribution of such occurrences may be used to establish normal, edge, and outlier cases. In a merely exemplary embodiment, a normal case may be within a standard deviation of a median value in the normal distribution while edge cases are between one and two standard deviations and outlier cases are greater than two standard deviations from the median. The categorized cases, as explained in further detail herein, for example, identified edge cases may be further investigated to create or improve event detection algorithms, rules for clinical decision support, alert algorithms, and predictive algorithms.

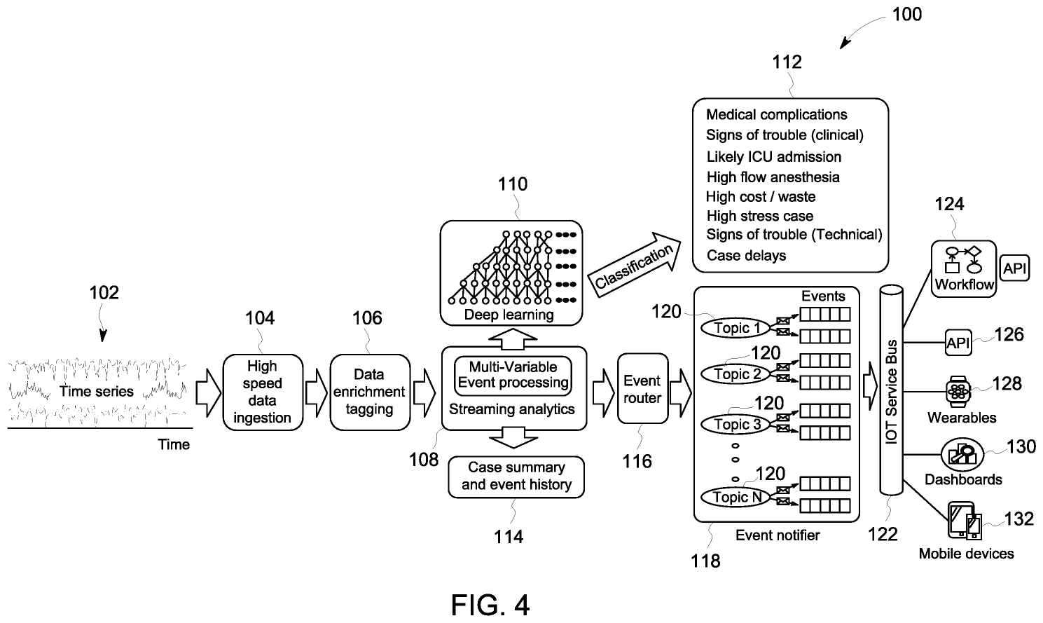

The stream processing engine 28 also identifies events in the time series streams of medical device data, for example in the manners as described in further detail herein and presented in business intelligence and visual analytics tools 32 which exemplarily may be presented on a graphical display communicatively connected to the medical device data processing system 12. The processing and reporting of events will be described in further detail herein with respect to FIG. 4.

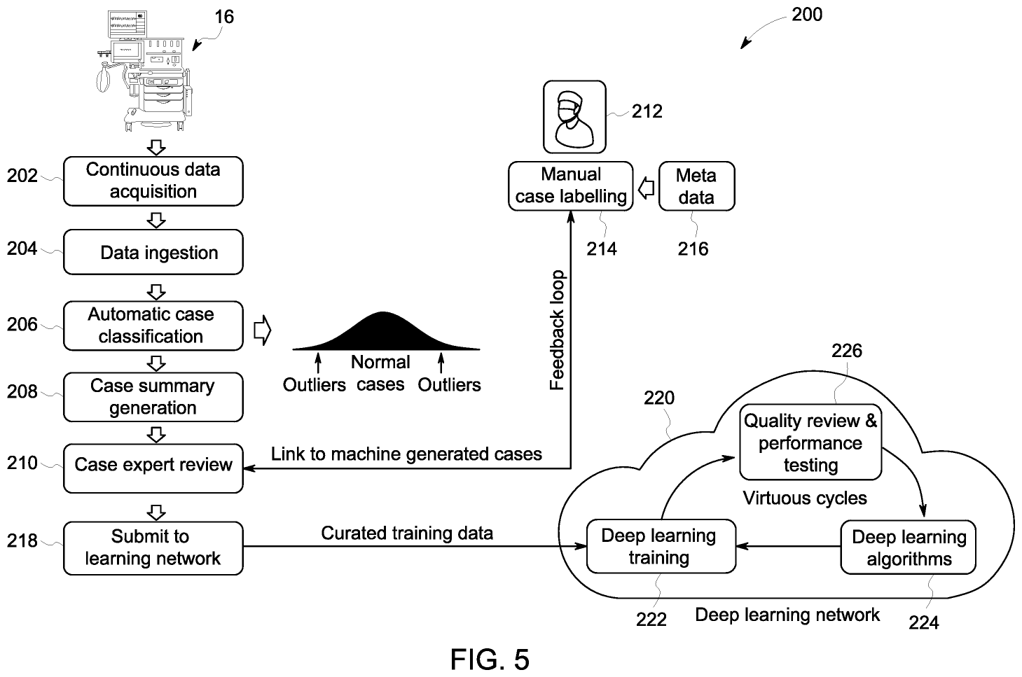

Once clinical cases are stored in the operational case database 30, clinical cases can be reviewed manually by a clinician or technician using a curation and case review tool. The curation and case review tool 34 can be presented in a graphical user interface on a graphical display and further provide inputs exemplarily through the graphical user interface for the user or technician to curate or otherwise assess the clinical cases. This can be performed for investigative, educational, and data curation purposes. Exemplary and non-limiting embodiments of the data curation will be described in further detail herein with respect to FIG. 5.

The reporting and visual analytics tool 32 can present the detected events in a variety of channels of communication. Exemplarily as will be explained in further detail herein, and including, but not limited to, the detected events can be presented visually through graphical user interfaces and graphical displays. The detected events or notifications of the detected events can also be reported by communication of events/event notifications to wearable or mobile devices and presentation of medical device data and identified events in visual form in reports and/or dashboards presented in a graphical user interface on a graphical display.

The results of the streaming analytics and event detection in the time series of medical device data can be provided to an application programming interface (API) 38 for use by application developers to provide monitoring, reporting, and/or control applications based upon the analyzed streams of medical device data. Such applications may operate through a computer operating system, a website browser, or operate on a mobile computing device or wearable computing device. Non-limiting examples of applications that may leverage the analysis of the time series medical device data include, but are not limited to an anesthetic agent cost dashboard 40, a checkout dashboard 42, a supervisory application 44, an alarm management application 46, an asset management application 48, and a benchmarking application 50.

The agent cost dashboard 40 exemplarily presents medical device data regarding anesthetic agent use across clinical cases as well as between anesthesia delivery machines within a hospital network or comparatively between hospital networks. By comparatively presenting this information anesthetic agent use and behavioral changes can be understood and undertaken to promote efficient use of anesthetic agent.

The checkout dashboard 42 may exemplarily assist in monitoring the inspection and maintenance of the monitored medical devices. Medical device data such as device status and settings, as well as messages and information in machine data may provide insight into the inspection processes for maintaining medical devices at a hospital network. The checkout dashboard may identify maintenance and/or testing events in the streams of machine data and note these identified testing events against a testing schedule, requirement (e.g. daily), or other criterion.

A supervisory application 44 may be used by attending and/or supervising anesthesiologists to more efficiently manage remote personnel and/or nurse anesthetists simultaneously working across multiple locations or theatres. An alarm management 46 application can report and present medical device data regarding alarm notifications and silences of alarm notifications in order to better understand and adjust sheen alarms to improve signal to noise in alarm events and to reduce alarm fatigue by clinicians.

Asset management applications 48 may present use, status, maintenance, and/or inspection information regarding medical devices (e.g. anesthesia delivery machines) or consumables used by medical devices, including components which while not replaced at every use must be frequently replaced, refilled, or refurbished during normal operation of the medical device (e.g. filters, absorbers). Benchmarking application 50 may provide further operational and quality performance across providers and/or organizations or in a comparative manner for example between hospital networks versus averages or between specific locations. Particular events and the application will be described in further detail herein.

FIG. 4 is a flow chart that depicts an exemplary embodiment of a method 100 of processing medical device data. An exemplary embodiment of the method 100 may be carried out by the medical device data processing system 12 as described above with respect to FIG. 3. As previously noted, embodiments of the systems and methods as described herein process medical device data as time series data of changes in each medical device data value 102. The data streams are acquired from the medical devices and may include time series format data regarding machine data such as alarms, device status, settings and values representing specific actions, messages, and/or measured data. The medical device data may further include patient physiological data obtained by sensors of the medical device. At 104 the time series data streams, multiple streams from each medical device in a medical device network are ingested to prepare the incoming data for processing and/or saving. In one exemplary embodiment, the medical device data is machine data. At 106 the processed medical device data is enriched by tagging the streams of data with identifying information and/or converting units of incoming medical device data to normalize units. The data enrichment and tagging at 106 is performed without negatively impacting latency of the ingested streams of medical device data. Streaming analytics are undertaken at 108 with a clustered streaming analytics engine capable of complex event correlation, data smoothing, and windowing operations. The streaming analytics is exemplarily performed by predictive algorithms from deep learning as will be described herein and in further detail with respect to FIG. 5.

In streaming analytics, data may be windowed into short segments against which the pattern recognition or predictive algorithms are applied. Further analysis can be applied between windowed portions of the time series and the windows overlap so that data is not missed and each segment of time series data is given both forward and backward context. As noted above, the analysis of the streaming time series data may not be limited to analysis of individual streams of data. Rather, different streaming analytics algorithms may relate the analysis of multiple streams of data, for example streams of data originating from the same medical device and/or streams of data originating from different medical devices but from the same location. This context may be provided by the tags added in the previous data enhancement. In still further embodiments, streaming analytics algorithms may analyze the streams of data in combination with other information as may be acquired from sources outside of the medical devices themselves. This other information may include but is not limited to time, date, hospital scheduling information or maintenance schedules in order to further carry out the clinical case identification and event detection as described herein.

The streaming analytic algorithms may exemplarily be alarm algorithms, control algorithms, event detection algorithms, or predictive algorithms. These algorithms may be predefined, or in other embodiments may be created as a result of automated learning, for example in the manners as described in further detail herein with respect to FIG. 5. Non-limiting examples of the streaming analytics algorithms may include early detection of deterioration of a patient's health status, detection of complications in an operating room (OR) or ICU, prediction of a medical device needing maintenance, prediction of complications in cases which may require future special care, or prediction of schedule delays. In exemplary embodiments, the streams of machine data from the medical devices may reflect the manner in which the medical device is being used and from analysis of the operation of the medical device, an event such as a complication during a surgery may be detected based upon this machine data. As will be described in further detail herein, the prediction of such a delay can be leveraged to reorganize schedules or room assignments for later scheduled procedures. In still further embodiments, the streaming analytics algorithms can be used to predict a likelihood of admission to an intensive care unit from an operating room or to predict a likelihood of avoiding the ICU after a procedure in the OR. These predictions may exemplarily be based upon the manners in which the medical devices were used during the procedure in the OR as a result from analysis from the streaming machine data from these medical devices.

In still further exemplary embodiments, the streaming analytics algorithms may be used to detect deviations from standard practice and/or to detect cases completing ahead of schedule or with a delayed schedule. For example, these may be detected by an identification of an unusually long induction phase, maintenance phase, or emergent phase as determined from the operation of an anesthesia delivery machine as compared to normal or expected operation. In other exemplary embodiments the results of streaming analytics algorithms may further be used to reduce nuisance alarms and to prioritize other alarms or patient critical alerts. Other exemplary embodiments apply streaming analytics algorithms to provide benchmarking of quality metrics for completion and provision of the procedures provided in the clinical case. Additionally, streaming analytics algorithms applied to the machine data of the medical device data can be used to infer likely activities or events during the procedure and document the times at which activities occurs for procedure summary and reporting as well as to predict post procedure outcomes.

The streaming analytics at 108 may classify events detected in the time series medical device data. These identified events may be classified as reactive, predictive, or prescriptive. Reactive events are those events which have already occurred. Predictive events are the events with statistically ranked outcomes and prescriptive events are those predictive events which also include recommended actions. At 110 deep learning algorithms may further classify the events with more specificity than the above noted reactive, predictive, and prescriptive. In non-limiting examples, the deep learning algorithms may classify the events as medical complications, likely admissions to the ICU, adverse outcomes, operational delays, high cost/waste of medical resources, additional events may be classified which detect early signs of trouble which can be both clinical and technical in nature. Additionally, the detection algorithms may flag high stress cases in addition to identifying chronology of medical device use, including delivery of various anesthesia phases, machine checkout and maintenance events, or the prolonged practice of high flow anesthesia. As will be explained in further detail with respect to FIG. 5, the behavior and accuracy of the predictions improve over time with more data, patterns, and through self learning.

The deep learning at 110 can be used to produce algorithms 112 for example through the methods as described in further detail herein with respect to FIG. 5. These algorithms may in turn be used for streaming analytics. Non-limiting examples of algorithms which may be developed from the deep learning at 110, may include algorithms to detect medical complications, to detect signs of clinical trouble, predict likely ICU admission, detect high flow anesthesia, detect high anesthesia cost or waste, detect high stress cases, detect signs of technical trouble in the medical device, or predict delays in further scheduled cases in a unit based upon detected prior events.

A case summary module 114 maintains state information for the clinical cases and tracks associated events for particular clinical cases. The case summary module 114 further helps the streaming analytics 108 by identifying recent historical events to help the streaming analytics 108 from identifying the same events excessively in the time series data and further maintains identified events in the chronological order. For example, if a clinical case start has been identified, focus can be redirected to identifying a clinical case end, rather than continuing to identify a further clinical case start.

In exemplary embodiments the streaming analytics at 108 can detect various events occurring in the streaming medical device data. Non-limiting examples of detected events may include those events which may be detected from a single data stream, for example a change in gas flow provided by an anesthesia delivery machine, for example a transition between high flow to low flow. An event that a machine checkout by a technician occurred may be detected, for example by noting a signal that is input or activated during a part of a checkout procedure. Various machine operability signals may be monitored, for example related to operation of a microprocessor to indicate whether or not the machine is with or without power or is in an on, off, or stand-by state. Respiratory events indicating patient airway troubles may be detected from the streaming machine data indicative of respiratory support provided to a patient in the cases of respiratory assistance for a spontaneously breathing patient or a clinician change to the respiratory support provided by a machine in the case of mechanical ventilation. Still further exemplary events which may be detected in the machine data include alarm status changes which indicate alerts and/or critical alarms, or technical alarms. In additional embodiments, events in the process of a medical procedure may be detected in the medical device machine data. For example, the start of a surgical case by detecting that medical devices are turned on or initialized, the detection of the start of the delivery of anesthesia or another medical device providing therapy or patient monitoring, detection of induction, maintenance or emergence phases, or the end of a medical procedure.

In still further exemplary embodiments, complex event processing may be used to identify events which occur upon event detection based not only upon the analyzed streaming medical device data, but also may be detected in view of the combination of multiple streams of medical device data and/or trends in various streams of medical device data.

Events detected by the streaming analytics at 108 are provided to an event router 116 which routes the identified events into predetermined groups or topics in an event notifier 118. The event notifier 118 receives the identified events from the event router into a plurality of groups or topics 120. The groups may be filled with similar events based upon type of medical procedure, type of device, location of device, or type of event. For example, all of the events that are detected as originating from a particular hospital, or a particular OR may be grouped together for event notification. In another embodiment, all of the events related to a particular type of device e.g. an anesthesia delivery device, may be grouped together. In a still further example, any events detected related to anesthetic agent consumption may be grouped together. Within each group, the importance or severity of the events may be determined and a notification for the events produced accordingly. Low priority events may be stored to an event log for later review. While high priority events for example detected software or hardware malfunction, diagnostic or predictive events may be provided as disclosed herein to more directly alert responsible personnel.

A service bus 122 is connected to the event notifier and publishes the event notifications to the appropriate consuming endpoint depending upon the notification and the priority of the notification. Channels of transmission/communication of event notifications as well as personnel to which even notifications are communicated may be predetermined based upon the groups into which the event notifications are routed and/or services of the event notification. Notifications may be provided to workflow engines, for example as may be used to coordinate personnel and task workflow within a hospital at 124. Events may be directed to a push notification application programming interface (API) for sending and receiving push notifications to the identified recipient(s), for example on a wearable 128, a dashboard 130 on a laptop or desktop computer, or on a mobile device 132. In other embodiments, wearables, 128, dashboards 130, and/or mobile devices 132 may connect directly with the service bus 122, for example through an app or other program operating on the device that connects to the service bus 122.

FIG. 5 depicts an exemplary embodiment of a method 200 for data curation and deep learning. Exemplary embodiments of the method 200 may be carried out with the medical device data processing system 12 as described above with respect to FIG. 3. As depicted in FIG. 3 a plurality of medical devices 16 produce streaming time series of medical device data. The medical devices 16 may be located all within the same unit within a hospital, within the same hospital, or across a plurality of different hospitals and locations. The method 200 begins by continuously acquiring the streaming time series of medical device data at 202 from all of the medical devices. As noted above, the medical device data includes, but is not limited to data streams in time series format of machine data, for example alarms, device statuses, settings, messages, and data measured from device sensors. The medical device data may further include identified physiological data acquired by the medical device. These pluralities of streams of time series data from the plurality of medical devices are received by the medical device data processing system by ingesting the streams of data into the system to process the data for further processing and storage. At 206 the streaming analytics performed by the streaming processing engine 28 automatically detects clinical cases from the medical device data at 206. The clinical cases can be detected in the manners as described above. The identified clinical cases are further classified into normal cases and outlier cases. The identified clinical cases are saved, for example in the operational case database 30 and/or the data lake 26 with the summary of the case classification as normal or an outlier at 208. Edge cases and outlier cases are flagged at 210 for independent review by a clinician expert. The flagged cases are provided to clinician experts and are reviewed at 212 by the clinician expert. In the clinician expert review, the case summary of the identified clinical case is combined with the clinician's own manual case labeling at 214 and additional metadata 216 reviewed by the clinician in evaluating the flagged cases. The additional metadata reviewed at 216 may include the identified physiological information regarding the clinical case and/or events detected in that same clinical case. While at 218 the identified normal cases are submitted to the learning network at 220, the clinician expert has the option of submitting or withholding the reviewed edge or outlier cases from the learning network 220.

In a still further exemplary embodiment, identified clinical cases may further be evaluated and identified in a manner of case profiling. In exemplary embodiments, review and evaluation of clinical cases either automatedly or in a combination of clinician and automated review, can help to identify clinician work flows from the medical device data streams. For example, changes in anesthesia amounts during the course of a medical procedure can be identified and related to particular anesthesiologist techniques, work flows, or practices. For example, in one embodiment, anesthesiologist's use of anesthetic agent to control blood pressure may be identified while in other cases specific anesthesiologist's techniques or workflow, for example use of a “coasting maneuver” during an emergence phase may be detected.

Deep learning training 222 takes in new clinical cases and links the clinical cases and/or curated cases to the medical device data records in the data lake with each clinical case. The clinical cases and the associated medical device data are used by the deep learning network 220 to train deep learning algorithms 224. In embodiments, the deep learning network 22 may additionally incorporate curated data from a wide variety of clinical research institutions that have joined into a shared learning and discovering community. The deep learning algorithms 224 may be predictive algorithms and/or rules which define particular outcomes, predicted outcomes or other events. The deep learning algorithms 224 are quality tested at 226 against the clinical cases and medical device data representing those clinical cases as stored in the data lake of the system. The deep learning algorithms 224 can be provided to the stream processing engine for use in the streaming analytics as previously described. In exemplary embodiments, the streaming analytics 108 (FIG. 4), exemplarily by the stream processing engine 28 (FIG. 3) analysis of the machine data in order to identify ventilation mode usage, performance of recruitment maneuvers, use and level of PEEP, delivered tidal volume, as well as other measures of ventilation delivery and procedure effectiveness and use for example, oxygen saturation, hemodynamic values, end tidal gas concentrations, lung volume changes, and patient lung compliance. Relative values and/or changes in any of those metrics may also be used to identify between procedure phases.

In a non-limiting embodiment, an anesthesia delivery management system 18 locally connected to a plurality of anesthesia delivery machines 16b at a hospital 14 receives the medical device data from each of the anesthesia delivery machines 16b. In an exemplary embodiment, the anesthesia delivery management system 18 may calculate a vapor flow rate for each anesthesia delivery machine 16b as it is used during an identified surgical case. This calculation may be based upon information obtained and identified from machine data from each of the anesthesia delivery machines 16b. A stream processing engine operating, for example, on the anesthesia delivery management system 18 can detect the event of a surgical case beginning through the anesthesia delivery machine data time series streams, for example which may show the anesthesia machine powering up, going through an initial checkout process or cycle, and then the initiation of delivery of anesthesia by the machine. This may be used by the stream processing engine to identify the clinical event of a surgery using anesthesia. Additional medical device data streams from that anesthesia delivery machine or other medical devices, e.g. a patient monitor, associated with that anesthesia delivery machine may also be used to identify the clinical event. The stream processing engine 28 may further receive a calculated anesthetic vapor flow rate from the anesthesia delivery management system 18, or may receive the individual device settings of the vaporizer to enable calculation of anesthetic vapor flow rate. Anesthetic vapor flow rate can be calculated based upon a fraction of inspired agent and the fresh gas flow rate. Still further embodiments may additionally require the expired minute volume the inspired minute volume and a fraction expired anesthetic agent.

Based upon the settings of the anesthesia delivery device, an anesthesia setting identifying the anesthetic agent delivered to the patient can be provided to the system. From the identified agent type, a conversion factor can be applied to the anesthetic vapor flow rate to arrive at the liquid anesthetic flow. Liquid anesthetic flow is used against the current volumetric cost of the liquid anesthetic agent. From this calculation, an instantaneous, average, or total anesthetic agent cost can be calculated.

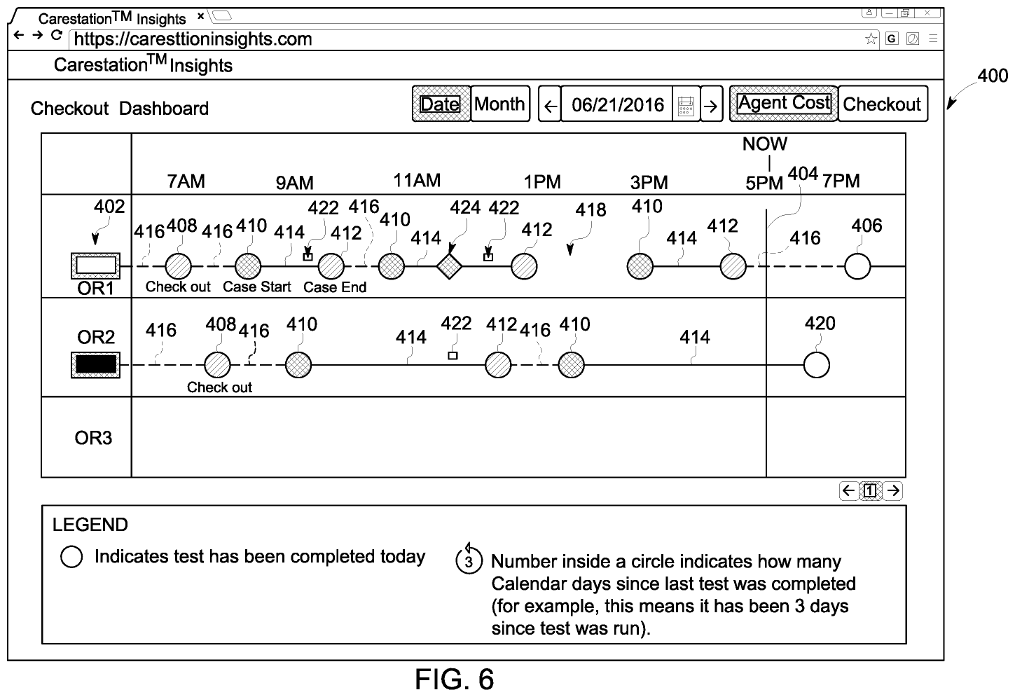

FIG. 6 depicts a further exemplary embodiment of a monitored room status dashboard presented as GUI 400. In the GUI 400, a temporal view of the use each of a plurality of monitored rooms 402 is reported to visualize both a current status of the use of an operating room, but also a future schedule of the operating room, this visualization can assist a clinician or clinical manager in staffing and/or scheduling management to estimate and/or reflect whether the operating room use is on time or behind schedule. In an event that actual performance of the clinical procedures are deviating from the schedule, accommodations may be made to move future procedures to another room or with alternative accommodations.

The GUI 400 exemplarily presents a temporal presentation with a line 404 indicating a current time. Future scheduled events 406 are presented to the right of the line 404 while the actual timing of the procedures as they occurred are presented to the left. The events of the procedures as they occurred can be detected by analysis of one or more streams of medical device data, including streams of machine data from multiple medical devices located in each of the monitored rooms 402. The analysis of machine data from multiple medical devices associated with a medical procedure can further improve accuracy of event detection by the correlated indications found in the machine data of these separate devices. In an exemplary embodiment, individual circles may indicate checkout procedures 408 while connected circles indicate the start 410 and ends 412 of various procedures. The operational status of machines or events may be indicated by a line. Exemplarily solid lines 414 indicate an active procedure while dashed lines 416 indicate a standby status. A gap or no line 418 may indicate when machines are off or a facility is otherwise closed. In an exemplary embodiment, when a procedure is currently ongoing, the system may produce an estimation 420 of a procedure end time. This estimation of procedure end time 420 may be exemplarily based upon the underlying schedule as well as any detected events in the medical device data during the procedure thus far that would indicate a delay in the estimated procedure end time. Detected events 422 may be indicated along the procedure which may indicate a prolonged procedure, or other departure from a baseline estimate of a procedure end time. Still further events, for example a secondary induction phase 424 may be indicated as such an event may further indicate a prolonged procedure. In exemplary embodiments, medical devices may be designed to incorporate additional sensors such as to further collect and report machine data as used in exemplary embodiments described herein. Such machine data may exemplary include flow sensors or valve cycles, processor clock speeds, component voltages. Changes in these signals may exemplarily be used to track a condition and/or “wearing out” of consumables and/or commonly replaced components of a medical device, non-limiting exemplary embodiments may include a CO₂ absorber, humidifier, filters, valves, or gas sensors.

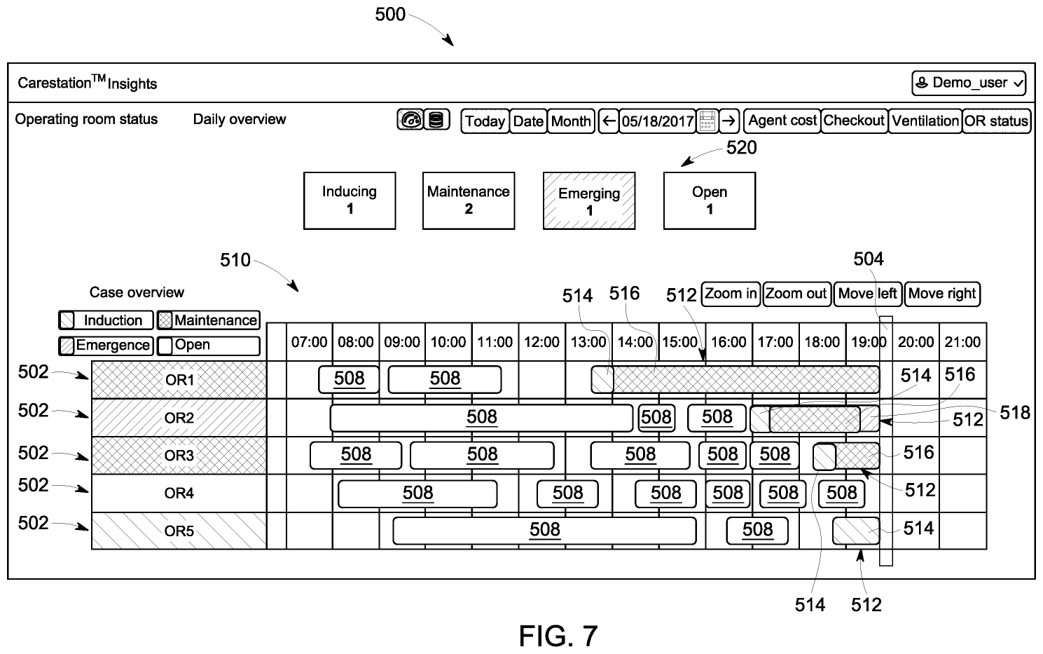

FIG. 7 depicts an exemplary embodiment of a monitored room status dashboard presented as GUI 500. The GUI 500 includes a table 510 that presents an overview of the past and current status of each of a plurality of monitored rooms 502, e.g. operating rooms. Each monitored room 502 is represented in a row in the table 510. The table 510 provides a temporal depiction of the status of the monitored rooms 502. A line 504 indicates the current time in the presentation of the table 510. Additionally, each row representing a particular room 502 includes indications of past procedures 508. These indications may be presented in a greyed, transparent, or lighter color to indicate that these procedures are not active and have been completed. The ends of the indications of past procedures 508 can coincide with a detected procedure start and/or completion times and the lengths thereof thus representative of procedure length.

The indications of past procedures 508 provide a record of the prior use status of each of the monitored rooms 502. The indicated past procedure 508 therefore also provides an indication of a past “in use” status, while the spaces between the indications of past procedures 508 provides an indication of a past “out of use” status. Additionally, the indicated past procedures 508 further provide a visual indication of the first case start time for each of the monitored rooms 502. The onset of the earliest record of an indicated past procedure 508 for that day is also when that monitored operating room 502 began being used for the day. Similarly, as no more procedures are performed for the day, a last case end time for each of the monitored operating rooms 502 will be visually presented from the latest indication of a past procedure 508. For example, looking to FIG. 5, if no further procedures are performed in OR4, then the last case end time for OR4 may be noted as 18:45.

The table 510 further identifies active procedures 512. The active procedures may be indicated in one or more colors or visual effects, for example to set them apart from the indications of past procedures 508. As described in further detail herein, the system may further operate to detect particular events or phases within the active procedure. While the indication of detected events or phases are described and displayed herein with respect to the active procedures 512, it will be recognized that in embodiments, such indications may additionally be provided relative to the indications of past procedures 508. For a patient receiving anesthesia, the anesthesia status of the patient may be determined to be in one of three anesthesia phases—induction, maintenance, and emergence. These are exemplarily represented within the active procedures 512 further with color, gradient, textural or other indications of the induction phase 514, maintenance phase 516, and the emergence phase 518 In an exemplary embodiment, the active procedures 512 are made up of bars that sequentially identify the phases of the current procedure as determined by the system as described above and in further detail herein. The start, end, and length of the phases further exemplarily coincide with the time identifications of the table 510. An operating room that is currently open or unused is exemplarily indicated as such with the absence of an indicated active procedure 512, for example, as is shown in FIG. 5 with OR4. The row associated with OR4 presenting only past procedures 508 and there being no current active procedure 512 in that row.

The GUI 500 operates to provide a real-time status of the current state of the use states of each of the monitored rooms 502. The GUI 500 also operates to visually present this information in other manners as well. In a first example, the GUI 500 illuminates an indication of the monitored operating room 502 with a color or other indication as noted above that is representative of the use status (e.g. anesthesia phase) of that monitored operating room. For example, the monitored room identifications 502 respectively for OR1 indicates that the room is currently used in an anesthesia maintenance phase, OR2 indicates that the room is currently used in an anesthesia emergence phase, OR3 indicates that the room is currently used in a anesthesia maintenance phase, OR4 is currently open, and OR5 is currently used in an anesthesia induction phase. The GUI 500 also provides a status summary 520 that indicates the total numbers of monitored operating rooms that are currently in use in induction, maintenance, emergence, and open use states.

As noted above, embodiments of the system operate to analyze MDD, for example taken from the anesthesia delivery device itself, as a source of de-identified data that can be refreshed and ingested at a high temporal resolution to provide both a real-time indication of the current use status of one or more operating rooms, but also for prediction of when future use status will occur within each monitored room. Such use status indications and/or predictions of future use status occurrences may require analysis of multiple streams of MDD. The current use status, procedure phases, or predictions of the occurrence times of future use statuses or procedure phases can be performed as described herein through analysis of the streaming time series MDD without further user or clinician input, and may also operate without input from other scheduling tools. However, as will be described herein, the output of such determinations may be inputs to available scheduling tools. In still further embodiments, the time duration for each procedure phase may be determined and used as an indicator or in a determination of when a subsequent procedure phase will begin or when the current use status of a monitored room will change.

MDD from the anesthesia delivery device can include values that are indicative of the presence of patient exhalation (e.g. exhalation pressure, tidal volume, expired gas concentration). As an anesthesia operating room use case can only occur when the patient is connected to an operating anesthesia delivery device, the connection/disconnection of the patient from the device (as determined from data that indicates patient exhalation) can provide a first-order indication of use status, or use start/end. From the determination that a use case has started, a current particular phase of use status can be determined next. Anesthesia typically includes three sequential phases of induction, maintenance, and emergence. By evaluating MDD received from the anesthesia delivery device, the system determines the current phase of the anesthesia. As previously noted, induction phase may be initially indicated by MDD indicating operation of the anesthesia delivery device and exhalation of the patient. Induction phase is further characterized by increasing flow rate of anesthetic agent.

FIG. 6 depicts an exemplary embodiment of a system 600 for processing medical device data (MDD). The system 600 depicted in FIG. 6 is exemplarily provided in two portions. A local portion 602 exemplarily occurring within the operating room (OR) or within another monitored room as described. While the example of an operating room is used herein, it will be recognized that in other embodiments, the local portion 602 may take place in another type of monitored room within a medical care facility, provided that such monitored room includes at least one medical device that produces MDD as described in the present application. Exemplarily, the monitored room of the local portion 602 includes a medical device of a patient monitor 604 and a medical device of an anesthesia delivery machine 606. It will be recognized that other medical devices may be used within the monitored room 602 and thus may operate in the manner as described herein while remaining within the scope of the present disclosure. One or both of patient monitors 604 and the anesthesia delivery machine 606 produce medical device data (MDD) as described above. The MDD 608 from patient data 610 which may also be produced by one or both of the patient monitor 604 or anesthesia delivery machine 606 in that the MDD 608 is de-identified in that the streaming time series of MDD is produced and provided without identifying information which may relate to the specific patient, but rather to the function and operation of the machine from which the MDD is provided. The patient data 610 in contrast includes identifying information of the patient and thus is provided in a secure manner to an electronic medical record (EMR) 612 and provide either in series or in parallel from the EMR 612 to a medical facility scheduling system 614.

The local portion 602 is distinguished from the processing portion 616 of the system 600. The processing portion may be implemented in a cloud based system or may be implemented locally to the specific monitored room. Still further exemplary embodiments may be implemented in various manners between these two embodiments, for example with processing remote from the monitored room, but at another location within the medical facility, or in a distributed processing network in which portions of the processing may be performed on different devices and locations.

The MDD 608 is provided to an MDD processing system 618 as previously described. It will be recognized that the MDD processing system 618 may be implemented on one or more computers and may include the features as described above or more or fewer features based upon that disclosure as will be recognized by a person of ordinary skill in the art. The MDD processing system 618 receives streaming time series of MDD from at least one medical device in one or more monitored rooms. The MDD processing system 618 analyzes these streaming time series of medical device data to detect procedural events in the time series MDD. Procedural events may be detected as described above, and may be done so based upon models of expected MDD values or occurrence's within a modeled or expected procedure.

For example, based upon the time series MDD, medical device start up, initialization, and set up procedures can be detected and noted to identify the start of a procedure. Once a procedure is started, the use status of the monitored room can be identified and a current phase of the use of the monitored room identified. With regularly defined phases to detect procedures in a monitored room, the progression and/or occurrence of a phase can be determined from the time series MDD, for example based upon gas flows or particular operations or settings performed on the one or more medical devices in the monitored room. The MDD processing system 618 thus provides indications of monitored room status at 620. These indications may include a current use status of the monitored room, including whether the room is in use or not in use while further status indications may include a procedure phase of a current use. The indications of monitored room status at 620 can be used in various ways, including as an independent real-time feedback input to the scheduling system 614. In still further embodiments, the monitored room status at 620 can be reported as electronic alerts, to workstation, mobile, or wearable devices, for example in the form of text messages, push notifications, or in-app notifications. Because the monitored room status is determined from de-identified machine data, such real-time indications of monitored room status may be used to update a broader range of scheduling or notification functions, including, but not limited to, scheduling of cleaning teams, maintenance or service personnel, or inspection/management checks. Additionally, the indications of monitored room status 620 may further include a predication of when either a current phase of a current procedure will end, or when the current procedure will end. Such estimation may be based upon modeling of records of previous similar procedures and generalizations from prior similar procedures, for example from detected events, values, or occurrences in the time series MDD. These estimations of the end or commencement of the current or a subsequent procedure phase or use status can be similarly provided to the scheduling system 614 for use in medical facility scheduling.

Additionally, the indications of monitored room status at 620 are recorded or otherwise documented at 622 to provide daily use metrics. In exemplary embodiments as will be described in further detail herein, by aggregating the determined current use statuses for the monitored room over the course of the day, a record of the use of the monitored room is constructed with de-identified data. This record of the use of the monitored room can provide valuable metrics for medical facility management and improvement of scheduling and monitored room use. Daily use metrics may include a clinical start time, a clinical end time, a utilization rate, a case volume, or an average turnover time.

In a still further exemplary embodiment, the medical device data processing system and method as disclosed herein may be implemented and carried out locally rather than in a cloud-based implementation. In such an embodiment, for example, the use status determination methods and dashboard as described herein may be carried out for example at the anesthesia delivery management system 18 (FIG. 1). In a non-limiting example of such an embodiment, the anesthesia delivery management system 18 may carry out the clinical case identification, event detection, and notification processes described above. The anesthesia delivery management system 18 may further perform the procedural phase and phase/use status completion or initiation estimates locally to each monitored room. In a non-limiting exemplary embodiment, the streaming analytics may rely on algorithms and/or rules supplied to the anesthesia management system 18 rather than relying upon a locally implemented deep learning process.

In other exemplary embodiments, the use status, current procedure phase, and completion/initiation estimations may be embodied in a plurality of rules and/or processes which may be compared to the detected clinical events or occurrences, as described above. Still further examples of clinical events or occurrences which may be detected in the time series MDD may be a change in anesthesia delivery, an increase or decrease in oxygen concentration, expired gas concentrations. Still other examples may include ventilation settings including PEEP or recruitment maneuvers. From the time series MDD, clinical events such as intubation, extubation, decruitment, or suction may be detected.

FIG. 7 depicts an exemplary embodiment of an operating room summary dashboard presented at GUI 700. In the GUI 700, the daily use metrics as described above are presented for each of a plurality of monitored rooms 702. The table 704 presented in the GUI 700 identifies each of the monitored rooms 702 and further provides various monitored room use metrics as described above. The table 704 exemplarily reports the case volume 706, the clinical start time 708, the average turnover time 710, and the utilization rate 712 for each of the monitored rooms 702. The case volume 706 is the number of cases handled in that monitored room during the selected day or other time period. The clinical start time 708 is the time at which the first case started for the day in the monitored room. The turnover time 710 is the average amount of time between the completion of a clinical case and the start of a subsequent clinical case in the monitored room. The utilization rate 712 is the percentage of time between the start of the first clinical case and the end of the last clinical case for the day in that monitored room during which a case is performed as opposed to turnover time.

The GUI 700 further may present the current use status 714 of the plurality of monitored rooms. As an exemplary embodiment, it will be noted that the current use status 714 in the GUI 700 matches the current use status for each of the monitored rooms present in GUI 500. The GUI 700 further presents monitored facility metrics in which the metrics as presented in the table 704 and as previously described are additionally presented in an aggregate form combining such use metrics for all of the monitored rooms in the medical facility for a selected date or date range. Exemplarily, at 718 a selection may be made between presenting such monitored facility aggregate metrics 716 for a specific date or for a selected month at 718. Additionally, the monitored facility metrics 716 may include a comparative trend for example an indication of an increase or a decrease of each of the presented metrics over a trend time selected at 720. The GUI 700 exemplarily presents a 30 day trend as selected at 702.

FIG. 30 is a flow chart that depicts an exemplary embodiment of a method 800 of monitoring a use status of a medical facility. The method 800 begins with receiving streaming time series medical device data (MDD) from at least one medical device in each of one or more monitored rooms of a medical facility at 802. As previously noted, the time series MDD includes de-identified information and values from the medical device regarding the operation of the medical device. Such streaming time series MDD may include medical device input or settings or the values read by sensors incorporated with the medical device. In an exemplary embodiment, the time series MDD includes at least one, some, or all of the following MDD: fresh gas or component gas flow rate(s), anesthetic agent concentration delivered or exhaled, CO2 exhalations by the patient, ventilation system pressure, ventilation system flow, delivered volume and/or breath rate.

Next at 804, procedural events are detected in the MDD. The procedural events detected in the MDD may include changes in medical device operation and which may be indicative of a current use or operation of the medical device. Such events may include the initialization or set up procedure of a medical device, a change or initiation in gas flow rates, a change in system pressure for example due to connection or disconnection of the patient from a ventilation system, delivery or weaning of anesthetic agent concentrations, an oxygen flush, or other medical device operations.

A current use status of the monitored room is determined from the detected procedural events at 806. In an exemplary embodiment, the current use status of a monitored room includes an indication if the monitored room is in use for a clinical procedure or not in use for a clinical procedure. Such determination of the current use status of the monitored room may also include a determination of a use status start time and a use status end time, for example by way of noting a time at which the current use status of the monitored room changes. The current use status of the monitored room may be determined at 806 from the detected procedural events in the MDD. In an exemplary embodiment, on going detection of procedural events in the MDD may be indicative of ongoing current use of the monitored room while particular detected procedural events may indicate the start of a clinical procedure use or the end of a clinical procedure use.

At 808, a current procedure phase is determined. If a monitored room is determined to be currently used in a clinical procedure, then at 808 a current procedure phase of that clinical procedure may further be determined based upon detected procedural events in the MDD. In an exemplary embodiment, the current procedural phase may exemplarily be one of an induction phase, a maintenance phase, or an emergence phase of anesthesia of a patient. The current procedure phase may be determined at 808 for example from an analysis of the streaming time series MDD to detect procedural events in the streaming time series MDD relative to the procedure phase determined. Such procedural events may include, but are not limited to, changes in anesthetic agent flow rate, changes in oxygen flow rate, expiration pressure, or expired gas concentration. In an exemplary embodiment, the start of a clinical procedure and an induction phase may be determined from an increase in anesthetic agent flow rate from a previous zero flow rate. In a still further exemplary embodiment, an emergence phase may be identified based upon an anesthetic agent flow rate of zero combined with an increased oxygen flow rate.

In an exemplary embodiment, the transitions or initiation of each procedure phase may be indicated by a defined or modeled event between the value or values of multiple streams of time series MDD. As previously noted, machine initialization or set-up processes in the MDD are indicative of a procedure start. The procedure start may further include an increase in system pressure or the detection of CO2 exhalation by the patient. Based upon historical procedure data, an estimate of the start of the induction phase can be made in view of ongoing MDD that confirms set-up without a detected induction phase. A high flow rate of delivered gas and the start of anesthetic agent delivery may then indicate the start of the induction phase. In an exemplary embodiment, a high flow rate may be greater than 5L/min. Once the patient is in the induction phase, an estimate of the onset of the maintenance phase may be made. This may be based upon historical procedure data providing duration information for induction phases, the models of which may be refined as induction phase is monitored and/or progresses. For example, as patient exhaled agent concentration increases, the patient may be determined to be completing induction. Also, in embodiments, as the current duration of a procedure phase increases, the estimated completion time of that phase will also increase to reflect the elimination of earlier-completed procedures. For example, if a patient has already been in an induction phase for five minutes, then the odds that the induction phase will take longer will increase compared to the population of procedures, because shorter length induction phases have been eliminated from the potential outcomes.

In an exemplary embodiment, detected procedure events of reduced fresh gas flow rate and/or reduced delivered anesthetic agent may be indicative of a maintenance phase. In an exemplary embodiment, the reduced fresh gas flow rate may exemplarily be a value reduced from the induction flow rate and may further exemplarily be a value below 5L/min. Continued delivery of a consistent fresh gas flow rate and anesthetic agent concentration may be determined as a continued maintenance phase and an estimate of maintenance phase completion based upon comparison to historical procedure data or models based upon a plurality of patients' historical procedure data. In additional embodiments, an event such as a change in anesthetic agent delivered and/or a reduction of the fresh gas flow rate to a further reduced level, for example below 2L/min may be indicative of continued maintenance phase, but may be used to refine the estimate of when the maintenance phase may be completed and the emergence phase begins. In exemplary embodiments, the subsequent delivery of a second anesthetic agent or the use of low fresh gas flow rates may be indicate of a lengthened or a shortened maintenance phase, for example depending upon the specific values of the MDD and the historical procedure data. Events such as the end of anesthetic agent delivery and the provision of a high fresh gas flow rate and/or a high oxygen concentration flow rate are indicative of the onset of the emergence phase. The emergence phase may persist and be monitored with an estimate of the completion of the emergence phase based upon emergence phase duration, and/or exhaled anesthetic agent concentration. Finally, an event such as system depressurization, an end of the flow of fresh gas, and/or no further detected CO2 exhalations can each be indicative of the procedure end.

At 810, the current use status and/or current procedure phase may be presented to one or more users and/or clinicians for example in a visual presentation through a graphical user interface configured as a medical facility status dashboard as previously described.

At 812, an estimate of a use status or procedure phase timing is made. The estimate of the use status or procedure phase timing at 812 includes an estimate of when the current use status will end or the current procedure phase will end. Relatedly, the estimate of the use status or procedure phase timing may be an estimate of a time at which a subsequent use status or procedure phase may start. In an exemplary embodiment, such estimate of use status or procedure phase timing may be based upon the recent detected procedural events in the MDD and timing models generated from analysis of prior clinical procedures to provide an estimate of the timing of a future use status or procedure phase from the historical records of clinical procedures and the detected procedural events. In an exemplary embodiment, an emergence phase may be estimated to take ten minutes and therefore upon the determination of the start of an emergence phase, that emergence phase is estimated to be completed ten minutes later. However, while this estimate may be updated during the emergence phase if, for example, a procedural event such as an oxygen flush is detected after only a first minute of the emergence phase and based upon the timing models, the emergence phase is complete six minutes after an oxygen flush. Therefore, the estimate of when the emergence phase will be complete may be updated to reflect this new detected procedural event.

At 814, determined current use statuses, determined current procedure phases, and estimates of use status and/or procedure phase timing may all be communicated at 814 to a scheduling system for the medical facility. As previously described, the scheduling system may coordinate the scheduled use of the monitored room, subsequent rooms, and/or medical facility personnel needed to turnover a monitored room or perform a next clinical procedure in the monitored room. The provision of these current status and procedure phase as well as timing estimate developed in real-time to the scheduling system can supplemental the scheduling information provided to the scheduling system by other means within the medical facility.

Finally, at 816, the determined current use status and current procedure phases may be aggregated and documented over the course of a predetermined time period, for example over a time period of a day, a week, or a month. When calculated over the time period of a day this calculates daily use metrics. The daily use metrics, which may alternatively be over another predetermined time period, can be used within a medical facility to promote efficient use of monitored rooms. The daily use metrics may include, but are not limited to clinical start times, clinical end times, case volume, utilization rate, and turnover time. These daily use metrics may be stored for later analysis and/or visual presentation to medical facility personnel.

In exemplary embodiments, the use status, procedure phase, and timing estimates as described above and as would be recognized by a person of ordinary skill in the art in view of the above disclosure may be provided to the system operating to perform the method 300 and exemplarily as further used in the above-disclosed streaming analytics as previously hard coded (or manually coded) rules. These rules may be provided upon an initialization or set up of the system operating to perform the method 300, or may exemplarily be provided or updated through a software update to the system or a portion thereof. In other embodiments, the use status, procedure phase, and timing estimates and/or other rules used in the streaming analytics may be developed and provided by machine learning algorithms that develop the use status, procedure phase, and timing estimates and/or other streaming analytics rules through training and/or analysis of case data as described above.

As previously discussed, improved streaming analytics algorithms, for example for event detection, parameter value detection, or patient response detection may be created over time through the use of deep learning analysis of the machine data and patient data regarding use status, procedure phase, and timing estimates in the manner as described above. Additionally, such techniques may also be used to improve the algorithms and/or analysis performed to provide improved determinations of use status, procedure phase, and timing estimates.

In exemplary embodiments, analysis of machine operational status (e.g. in use, standby status, and offline) may further provide information and insights not only to a hospital to assist in efficient management of medical device assets, but may further provide generalized information regarding specific use case and use trends to provide insight into field use of medical devices, in still further exemplary embodiments, maintenance, use, or other schedules may be based upon utilization reports specific to particular devices.

In a still further exemplary embodiment, the analysis of the streams of medical device data may further facilitate clinical record keeping and report management for a particular medical procedure. In an exemplary embodiment, analysis of the machine data can yield a summary report that indicates the time of use, duration of use, anesthetic agent delivered, as well as a log of anesthesia delivery events, inputs or changes in the anesthesia delivery. As this information can be detected in the streams of medical device data, such a report and/or summary can be automatedly produced and electronically provided to a clinician for review and approval and/or revision and the annotation with additional comments. In an exemplary embodiment, the use status, procedure phase, and timing estimates may be associated to medical billing codes and a provisional medical billing report provided to the clinician or another hospital personnel for review and documentation or use in billing submissions. In an exemplary embodiment, the electronic case summary report may be expressed in the HL7 clinical document architecture standard for example to be incorporated into a patient's electronic medical record and/or to facilitate use for billing documentation.

In an exemplary embodiment, the streaming analytics of the time series medical device data can include a combined analysis of machine data and physiological data related to the same patient in real time or near real time. Upon evaluation of this combined information the patient condition and current device settings and operation can be determined. Based upon this determination, particular events may be identified, these events being recommended changes to medical device settings or parameters. This may include adjustments to any of the values in the received machine data or other identified medical device settings, including alarm parameters. In one exemplary embodiment, these events result in notifications to a responsible clinician with recommendations for such medical device setting changes. In other exemplary embodiments, the medical device data processing system communicates back to the medical devices, for example through the gateway 20 to provide operational instructions and/or medical device setting adjustments directly to the medical device. In a still further exemplary embodiment, such recommended actions may first be presented to a clinical or technician for approval and/or confirmation prior to the MDD processing system providing such instructions directly to one or more medical devices.

Exemplary embodiments of the systems and methods as described herein gather machine data produced by a plurality of medical devices in real time and at a high frequency approaching the frequency at which such machine data is produced by the medical devices. Streaming analytics of this machine data can yield large amounts of machine use and medical procedure data that, in exemplary embodiments is, by its nature de-identified. This procedure data can be summarized for improved retrieval and access at a later date, but stored in its raw form for use in data analytics and machine learning to automatedly produce are and improved algorithms exemplarily for use in the streaming analytics of the medical device data.

Embodiments of the systems and methods as described herein improve upon existing health information systems/electronic health records data as that data is typically focused on patient physiological data and reported and/or recorded at longer time intervals. The low frequency of this data limits the types of clinical decision support that can results from these sources of data. For example reduced data resolution can lead to inaccuracy in detection of minimum and maximum values and transient events/values in the collected data. Furthermore, the use of patient physiological data introduces concerns and/or challenges of maintaining such information in a confidential and/or de-identified state.

In the above description, certain terms have been used for brevity, clarity, and understanding. No unnecessary limitations are to be inferred therefrom beyond the requirement of the prior art because such terms are used for descriptive purposes and are intended to be broadly construed. The different systems and method steps described herein may be used alone or in combination with other systems and methods. It is to be expected that various equivalents, alternatives and modifications are possible within the scope of the appended claims.

The functional block diagrams, operational sequences, and flow diagrams provided in the Figures are representative of exemplary architectures, environments, and methodologies for performing novel aspects of the disclosure. While, for purposes of simplicity of explanation, the methodologies included herein may be in the form of a functional diagram, operational sequence, or flow diagram, and may be described as a series of acts, it is to be understood and appreciated that the methodologies are not limited by the order of acts, as some acts may, in accordance therewith, occur in a different order and/or concurrently with other acts from that shown and described herein. For example, those skilled in the art will understand and appreciate that a methodology can alternatively be represented as a series of interrelated states or events, such as in a state diagram. Moreover, not all acts illustrated in a methodology may be required for a novel implementation.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A system for monitoring a use status of monitored rooms in a medical facility, the system comprising:
a plurality of medical devices located in the monitored rooms, wherein each of the plurality of medical devices comprises at least one sensor that produces machine data corresponding to operation of that medical device, and wherein the plurality of medical devices are configured to output a plurality of streaming time series of medical device data that includes the machine data produced by the at least one sensor;
a central input-output module that communicates with the plurality of medical devices and receives in real time or near real time the machine data therefrom;
a central processing module in communication with the central input-output module, wherein the central processing module is configured to analyze the machine data to identify portions corresponding to occurrences of clinical cases via at least one of: detecting changes in operational status within the plurality of medical devices, detecting changes to devices settings within the plurality of medical devices, and detecting a delivery or consumption of anesthetic agent by the plurality of medical devices, wherein the central processing module is further configured to analyze the identified portions of each of the machine data corresponding to the clinical cases to identify procedural events within the identified portions, and wherein the central processing module is further configured to automatically determine a current use status for each of the plurality of monitored rooms of the medical facility based on the procedural events identified within the machine data; and
a display device that communicates with the central processing module and displays the current use status for each of the plurality of monitored rooms determined thereby;
wherein the central processing module is further configured to compare the current use status determined for each of the plurality of monitored rooms to expected use status corresponding thereto, and to generate an alert when the current use status is inconsistent with the expected use status.

2. The system of claim 1, wherein the plurality of streaming time series of medical device data comprises a plurality of time series of medical device data streams from a single device of the plurality of medical devices, and wherein the central processing module is configured to concurrently analyze the identified portions of each of the plurality of time series of medical device data streams corresponding to the clinical case from the single device to identify the procedural events and the current use status of the monitored room comprising the single medical device.

3. The system of claim 1, wherein the display device displays the use status for each of the plurality of monitored rooms in real time.

4. The system of claim 3, wherein the current use status further comprises a current procedure phase identified by the central processing module.

5. The system of claim 3, further comprising a memory module in communication with the central processing module, and further comprising clinical case models stored within the memory module that include estimated times for completing the clinical cases, wherein the central processing module is further configured to estimate a time at which the current use status will end based on which of the estimated times corresponds to the clinical case identified.

6. The system of claim 1, wherein the machine data is one or more of a compressor speed, a compressor cycle time, an internal pressure, a voltage, a clock speed, and a temperature.

7. The system of claim 6, wherein the machine data is received at or near a frequency at which the machine data is produced by the medical device.

8. The system of claim 7, further comprising a memory module in communication with the central processing module, and further comprising clinical case models stored within the memory module that include estimated times for completing the clinical cases, wherein the central processing module is further configured to determine a current procedure phase of the current use status from the procedural events, wherein the display device is configured to provide a real-time visual output of the current procedure phase, and wherein the central processing module is further configured to determine a predication of when a next procedure phase will begin.

9. The system of claim 8, wherein the central processing module is further configured to provide the prediction to a scheduling system for the medical facility.

10. The system of claim 9, wherein the central processing system is further configured to, based upon the prediction, communicate to the scheduling system for the medical facility to reserve a subsequently required resource.

11. The system of claim 7, wherein the procedure phases include induction, maintenance, and emergence.

12. The system of claim 11, wherein the medical device data further comprises anesthetic agent flow rate, oxygen flow rate, expiration pressure, and expired gas concentration.

13. The system of claim 12, wherein the plurality of time series medical device data comprises an anesthetic agent flow rate, and wherein the central processing module is configured to analyze the plurality of time series medical device data by identifying a start of a procedure and an induction phase based upon an increase in the anesthetic agent flow rate from a zero flow rate in the time series medical device data, and by identifying an emergence phase based upon an anesthetic agent flow rate of zero combined with an increased oxygen flow rate in the time series medical device data.

14. The system of claim 1, further comprising a memory module in communication with the central processing module, and further comprising a plurality of procedure models stored within the memory module, wherein the central processing module, in analyzing the plurality of time series of medical device data, is configured to compare the plurality of time series of medical device data to the plurality of procedure models to identify the current use status of each of the monitored rooms.

15. The system of claim 1, further comprising a memory module in communication with the central processing module, further comprising configuring the central processing module to generate and store in the memory module procedure models as a composite of a plurality of previously recorded and annotated time series of medical device data from the plurality of time series of medical device data.

16. The system of claim 15, further comprising determining an estimate of when a current use status will end by comparing the streaming time series medical device data to the procedure models.

17. The system of claim 1, further comprising a memory module in communication with the central processing module, further comprising a plurality of case identification rules for applying to the plurality of streaming time series of medical device data, the plurality of case identification rules being stored in the memory module, and the central processing module being configured to apply the plurality of case identification rules and to produce an indication of a type of procedure in which the medical device is used.

18. The system of claim 1, wherein the central processing module is further configured to aggregate the determined current use status for each of the plurality of monitored rooms of the medical facility over a predetermined time period, and to output a summary of the determined use statuses for the predetermined time period.

19. The system of claim 18, wherein the central processing module is further configured to calculate a utilization rate for each of the monitored rooms based upon the determined use statuses for the predetermined time period.

20. The system of claim 1, wherein the central processing module is further configured to determine at least one daily use metric from the plurality of streaming time series of medical device data from at least one medical device located in a given monitored room among the monitored rooms, wherein the at least one daily use metric comprises a clinical start time, a clinical end time, a utilization rate, a case volume, or an average turnover time.
